(19) United States
(12) Patent Application Publication
(10) Pub. No.: US 2025/0241255 A1
(43) Pub. Date: Jul. 31, 2025

(54) PHILODENDRON PHOENIX PLANT NAMED ‘PHOENIX’

(71) Applicant: Natalie Roe, Umatilla, FL (US)

(72) Inventor: Natalie Roe, Umatilla, FL (US)

(21) Appl. No.: 18/427,437

(22) Filed: Jan. 30, 2024

(51) Int. Cl. A01H 6/10; A01H 5/12

(52) U.S. Cl. CPC A01H 6/10

(57) ABSTRACT

A new and distinct cultivar of Philodendron Phoenix named ‘Phoenix’ particularly characterized by its distinctive leaf appearance; large plant size; mostly open and outwardly spreading fuller plant habit; vigor and slow growth rate; less-freely branching; flat leaves; and long and wide petioles with long petiole sheaths.

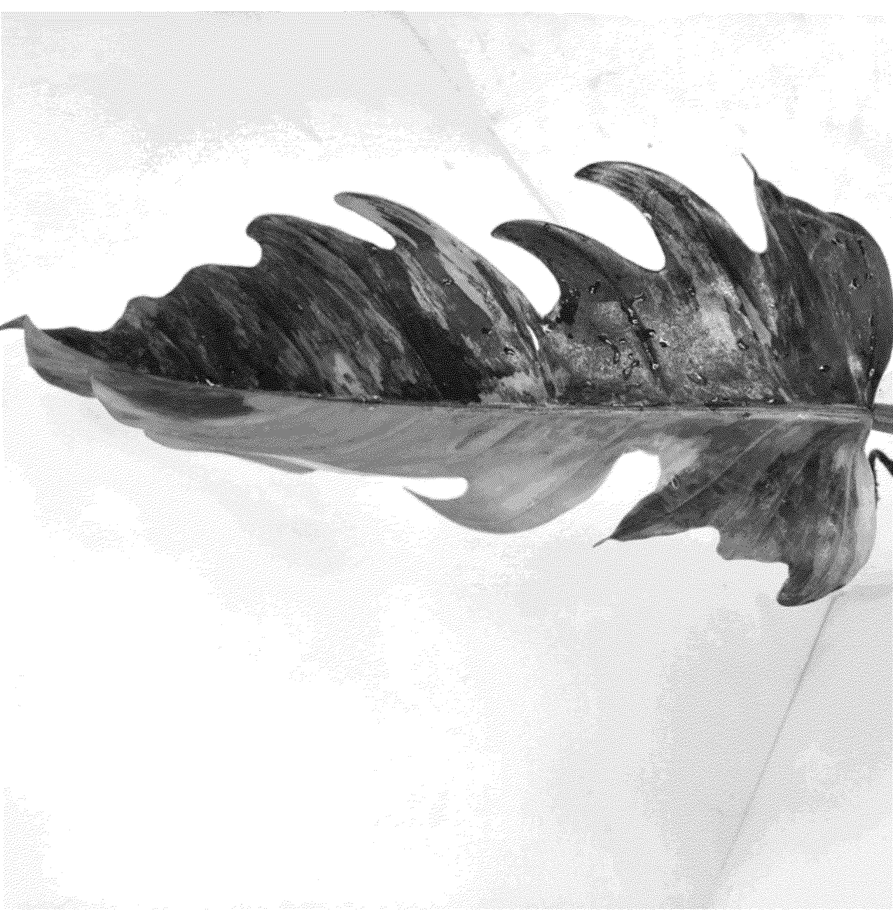

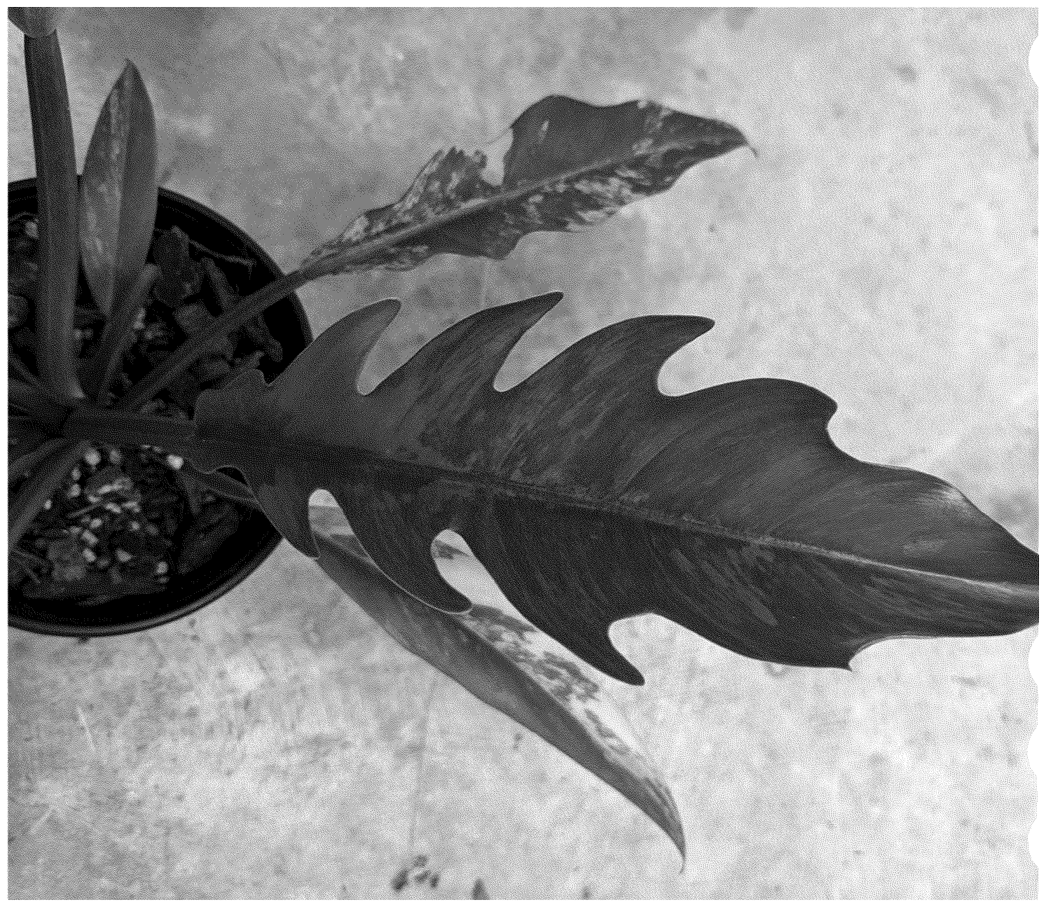

### CROSS-REFERENCE TO RELATED APPLICATION

The present application claims priority to U.S. Provisional Patent Application No. 63/441,887 entitled “PHILODENDRON PLANT NAMED ‘Phoenix’, filed Jan. 30, 2023. The disclosure of which is incorporated in its entirety herein by reference.

### BACKGROUND

The present invention relates to a new and distinct cultivar of Philodendron Phoenix plant, botanically known as Araceae Philodendron Phoenix, and hereinafter referred to by the cultivar name “Phoenix.”

The new Philodendron Phoenix is a product of an accidental breeding by the Inventor in Umatilla, Florida.

The new Philodendron Phoenix originated from an accidental breeding made by the Inventor, of an unidentified selection of the Araceae Philodendron Phoenix. The cultivar was discovered and selected by the Inventor, as a fully grown plant in an 8″ Growers Pot container within the progeny of the self-pollination in a controlled environment in Florida.

Compared to plants of the parent selection, plants of the new Philodendron Phoenix are larger, branch earlier, are less freely branching, and have a fuller plant habit and longer petioles.

Asexual propagation of the new Philodendron Phoenix by cuttings has shown that the unique features of this new Philodendron Phoenix plant are stable and reproduced true to type in successive generations.

### SUMMARY OF THE INVENTION

The new Philodendron Phoenix has not been observed under all possible environmental conditions. The phenotype may vary somewhat with variations in environment such as temperature, light intensity, fertilizer rate, and/or irrigation amount and frequency without any variance in genotype.

The following traits have been repeatedly observed and are determined to be the unique characteristics of ‘Phoenix.’ These characteristics in combination distinguish ‘Phoenix’ as a new and distinct cultivar.
1. Plants of the new Philodendron Phoenix are self-heading, not vining, and are self-supporting.
2. Plants of the new Philodendron Phoenix are relatively large in size with a mostly upright, somewhat outwardly spreading and open plant habit. Leaves are sharp-like with long round stems.
3. Plants of the new Philodendron Phoenix are vigorous and have a slow growth rate.
4. Plants of the new Philodendron Phoenix branch slowly and are early branching.
5. Plants of the new Philodendron Phoenix have large, thick, sharp-like, and flat leaves with orange, orange-pink, pink, green and red coloring.
6. Plants of the new Philodendron Phoenix have relatively long petioles and long, green petiole sheaths.
7. Plants of the Philodendron Phoenix have stems having green and red coloring. The stems are long and round.

Plants of the new Philodendron Phoenix can be compared to plants of the commercial Philodendron Phoenix cultivar Little Hope and Princes of Orange. However, in side-by-side comparisons conducted in Umatilla, Florida, plants of the new Philodendron Phoenix differ from plants of the cultivar Little Hope and Princes of Orange in the following characteristics:
1, Plants of the new Philodendron Phoenix are larger and are more outwardly spreading and open than plants of the cultivar Little Hope and Princes of Orange.
2. Plants of the new Philodendron Phoenix are less vigorous, form branches earlier and are less freely branching than plants of the cultivar Little Hope and Princes of Orange.
3. Leaves of plants of the new Philodendron Phoenix are thicker, more sharp in shape with truncate bases, and have more numerous primary veins than leaves of plants of the cultivar Little Hope and Princes of Orange.
4. Leaf color of plants of the new Philodendron Phoenix is orange, pink-orange, pink, red, green, and yellowish and there is a more striking contrast between the mid rib and the surrounding leaf tissue compared to leaves of plants of the cultivar Little Hope and Princes of Orange.
5. Plants of the new Philodendron Phoenix have longer and wider petioles than plants of the cultivar Little Hope and Princes of Orange. In addition, the shape of the petiole also differs longer, wider, and with green, yellow, and white markings. Some leaves may be pink/green colored white other leaves are bright orange-pink.
6. Leaf petiole sheaths of plants of the new Philodendron Phoenix are varying in color compared with leaf petiole sheaths of plants of the cultivar Little Hope and Princes of Orange. Plants of the new Philodendron Phoenix can also be compared to plants of the commercial Philodendron Phoenix cultivar Little Hope and Princes of Orange. However, in side-by-side comparisons conducted in Umatilla, Florida, plants of the new Philodendron Phoenix differ from plants of the cultivar Little Hope and Princes of Orange.

### BRIEF DESCRIPTION OF THE PHOTOGRAPHS

The accompanying color photographs illustrate the overall appearance of the new Philodendron Phoenix, “Phoenix’, showing the colors as true as it is reasonably possible to obtain in color reproductions of this type.

The photographs on sheets one through four comprise top perspective views of a typical plant of “Phoenix Philodendron” including immature leaves in a (size, e.g. 15 cm) container.

### DETAILED BOTANICAL DESCRIPTION

The following observations, measurements, and comparisons describe plants grown in Umatilla, Florida, under a polycarbonate-covered greenhouse and conditions which closely approximate those used in general, standard commercial horticultural practice. Plants were grown in fertilized soil under day temperatures ranging from 25 to 32 C and night temperatures ranging from 22 to 27 C. Light level ranged from 2000 to 2,2000 footcandles.

Plants used for this description were asexually reproduced by taking cuttings (twigs), rooted and planted into fertilized soil in 15-cm containers. Information was collected for this description and the cultivar comparisons in May 2022, when the plants were about 2 months old from taking cuttings (twigs). Numerical measurements represent averages from typical plants. Environmental conditions were tropical, humid, and required less water than typical Philodendron Phoenix.

In the following description, color references are made to The Royal Horticultural Society Colour Chart except where general terms of ordinary dictionary significance are used.

Botanical classification: Araceae Philodendron Phoenix cultivar “Phoenix Philodendron”.

Parentage: Self-pollination of an unidentified selection of Araceae Philodendron Phoenix.

Propagation: Type.—By taking cuttings (twigs).

Time to initiate roots.—Summer: About 21 days at about 25 to 30° C. Winter: About 28 days at about 22 to 27° C.

Time to develop (Time to produce a rooted liner).—Summer: About 10 to 12 weeks at about 25 to 30° C. Winter: About 13 to 14 weeks at about 22 to 27 C.

Rooting habit.—Strong root system. Main roots thick; abundant lateral roots. Thick short aerial roots are occasionally observed.

Plant description:

Growth habit.—Self-heading, not vining, and self-supporting. Mostly upright, but outwardly spreading, open, freely branching, Somewhat asymmetrical; mature plants, more sharp in overall shape. Appropriate for 15 to 25-cm containers.

Plant Size.—Height, Soil Surface to top of leaf canopy: About 50.7 cm. Width: About 99.2 cm.

Plant vigor.—Vigorous, slow growth rate.

Crop time.—About 48 to 52 weeks are required to produce a 40 to 50-cm tall plant in a 15-cm container from a 1 to 2-cm taken cutting (twigs).

Foliage description.—Shape of leaves on young plants: Between elliptic and ovate; apex, acuminate with some acute tendencies, base, obtuse; margin, entire. Shape of leaves on older plants: Ovate, apex, acuminate with Some acute tendencies, base, truncate; margin, entire. Length, mature leaves: About 36.7 cm. Width, mature leaves: About 18.8 cm. Length to width ratio, mature leaves: About 1.95 to 1. Aspect: Almost flat; Slightly concave at the petiole attachment. Texture: Thick, leathery, smooth. Surface: Glossy; young leaves very glossy. Color: Young, upper surface: At unrolling: Slightly brighter than 144 A. Once unrolled: Much brighter than 144 A. Young, lower Surface: 144 A. Mature, upper surface: Brighter than 147 A. About 1 mm wide band along margin, green/yellow or orange/pink in color, 144 A. Mature, lower surface: Brighter, including orange, pink, green, and yellowish 144 A, with subsequent development, leaf becomes slightly brighter and than 147 B. Venation, upper Surface of mature leaves. Primary veins are slightly lighter green than surrounding leaf tissue. Venation, lower surface of mature leaves: midrib and primary veins, mostly 146 C. Petiole aspect: Erect to semi erect. Petiole shape: Rounded lower Surface; upper Surface Slightly concave. Petiole length, primary shot: About 43.4 cm. Petiole diameter, just below leaf blade: About 10.3 mm. Petiole sheath length: About 21.2 cm. Petiole color, young leaves: 144 A to 146 C with short green, red, pink, and orange striae. Petiole color, upper Surface of mature leaves: Adjacent to leaf blade, close to 146 A to 146 B with Some 144 A, proximally lighter to 146 A to 147 B; slightly lighter near base. Petiole color, lower Surface of mature leaves: Adjacent to leaf blade, close to 146 B to 146 D and 14.4 A to 144 B; proximally darker, close to 146 A, base, close to 144 A. Petiole sheath: 144 B and 144 D with brightly colored striae.

Inflorescence.—Typical of Philodendron Phoenix, no commercial significance. Plants typically form inflorescences about 18 to 23 months after planting of a twig taken from cutting. Description of inflorescences taken from plants grown for about 2.5 years after planting a twig taken from cutting.

Arrangement.—Spathes with spadices with one to three inflorescences in each floral sympodium. Each developing inflorescence enclosed by two sheaths, which deteriorate before spathe opens. Spathes erect, situated near shoot base on short, thick, usually bent peduncles. Spathes typically tightly tolled around spadices, Spathes typically open for about one day. When spathes are open, the spadix is fully exposed, leaning forward beyond spathe margins, at about 50 to penduncle axis.

Fragrance.—weak, Similar to other Philodendrons, but weaker.

Peduncle.—Length: About 7.3 cm. Diameter: About 1.5 cm. Color: 144B or between 144B and 144C.

Spathe.—Shape: Boat-shaped; longitudinal section elliptic; apex between acuminate and cuspidate; base, obtuse; margin, entire and usually rolled back especially distally. Length: About 18.3 cm. Width: About 7.6 cm. Length to width ratio: About 2.4 to 1. Depth: About 4.8 cm. Texture: Very thick, fleshy, not glossy. Color: Open, front surface: Between 47 A and 47 B around spadix base, distally and toward margin, much brighter, 48 D to 49 D to close to 159 C near apex. Some yellow, red, orange, pink, and green 144 B, on midrib and along margins near apex. Open, rear surface: Most of the central Zone of proximal half is an uneven color, varying between different combinations of 146 C, 146 D, 144 A, 144 B and 144 C. Color becomes brighter and more red, pink, yellow, and orange towards margins, near margins, between 145 C and 145 D and translucent. In the distal half, there is a band along midrib between 144 B, and 146 C and 146 D. Spathe color on either side of this band is variable, but much brighter in some areas, most areas more yellow than 145 C and more green than 150 C. Often there is a wide, translucent band on one side near the margin.

Spadix.—Shape: Cylindrical, Strongly tapering towards acute apex. Female Zone, Situated at the base, shorter than the male Zone and Separated by an intermediate Sterile Zone. Sterile and male Zones not clearly distinguished when Spathe is open. Spadices typically sessile. Length: About 17.1 cm; female Zone, about 4.8 cm; Sterile and male Zones combined, about 12.6 cm. Diameter: Female Zone, about 1.9 cm; sterile and male zones, about 1.8 cm proximally. Color: Female Zone, between 145 C and 145 D; sterile and male zones, slightly darker than 159 C. Pollen: Cream in color. Dehiscence about one to three days after spathe closes.

Seed production.—Seed production has not been observed. Typically spathes and spadices gradually deteriorate after spathe closure.

Disease tolerance: Resistance to pathogens common to Philodendron Phoenix has not been observed.

What is claimed is:

1. A new and distinct cultivar of Philodendron Phoenix plant named ‘Phoenix’, as illustrated and described.
